(19) United States
(12) Patent Application Publication
(10) Pub. No.: US 2017/0000385 A1
(43) Pub. Date: Jan. 5, 2017

(54) SMART WEARABLE DEVICE SYSTEM

(71) Applicant: GUANGDONG COSONIC ACOUSTIC TECHNOLOGY CO., LTD., Dongguan (CN)

(72) Inventor: Liang Chen, Dongguan (CN)

(21) Appl. No.: 15/038,728

(22) PCT Filed: Dec. 25, 2013
(86) PCT No.: PCT/CN2013/090474

§ 371 Date: May 23, 2016

(30) Foreign Application Priority Data
Nov. 27, 2013 (CN) 201310610596.2

(51) Int. Cl. A61B 5/11; A61B 5/0205; A61B 5/0476; A61B 5/00

(52) U.S. Cl. CPC A61B 5/1113; A61B 5/0017; A61B 5/02055; A61B 5/0476; A61B 5/6801; A61B 5/742; A61B 5/0024; A61B 5/021

(57) ABSTRACT

A smart wearable device system includes at least two wearable devices having a health parameter test module, an anti-lost terminal module and a communication interface; and a smart control device having a health parameter control module, an anti-lost control module, a display screen and a communication interface. Each wearable device is wirelessly connected to the smart control device, and every two wearable devices are wirelessly connected to each other. The health parameter test module transmits physical health parameter information to the smart control device, the health parameter control module performs computational processing, stores and sends image information that reflects body health conditions, and the display screen displays the image information. The anti-lost terminal module has a location and time feedback module, and the anti-lost control module has parameter setting module and a location and time receiving module. The system can monitor physical health and prevent persons from getting lost outside.

### FIELD OF THE INVENTION

The present invention relates to smart wearable device system.

### BACKGROUND OF THE INVENTION

In prior art, it's common to use medical equipments to monitor physical health in hospital emergency room, laboratory, intensive care unit, for example to test body temperature, pulse and electrocardiogram, and the like. However, the equipments are complicated, the treatments take up much of time, and the cost for the treatment is very expensive, which are not desirable by the patient, as a result, some long-term monitoring treatments may not be carried out at some remote areas where the medical resources and physicians are limited. Research report of human health made by World Health Organization shows that, chronic diseases such as heart disease, hypertension, stroke, cancer, migraine headaches and diabetes currently are the biggest killer causing human death, while an omen of these diseases often is the so-called sub-health problem.

In view of the issues, people desire a medical system that is portable, room-saving and cost-low to monitor health parameters reflecting the chronic diseases. Chinese Patent Application No. 201220486834.4 discloses a wearable health healthcare intelligent device having a wearable micro-sensor that is connected with a smart phone by Bluetooth, and the smart phone is connected to network. Such a device is portable and takes up less room, and can monitor health parameters of the chronic diseases, thereby improving the popularity of the treatment and rehabilitation of the chronic diseases. However, drawbacks of the device at least include: first, the wearable micro-sensor is suitable for only one user; second, the function is simple, that is the device only can monitor body health conditions, but can not be served as a device that prevents persons from getting lost when they go out together.

### SUMMARY OF THE INVENTION

Technical problems to be solved in the present invention is to provide a smart wearable device system which can test health parameters such as blood pressure, oxygen saturation, blood sugar, pulse rate, electroencephalogram, body temperature and sweat, and send the parameters values to the smart control device to implement calculation processing and finally fed back to the user, further realize information transfer between multiple wearable devices, and efficiently realize the control, information transfer, interaction between the wearable devices and the smart control device (e.g. a mobile phone), and prevent persons from getting lost outside.

To solve the technical problems mentioned above, the technical solution of the present invention follows.

A smart wearable device system includes: at least two wearable devices, provided with a health parameter test module, an anti-lost terminal module and a communication interface which is electrically connected to the health parameter test module and the anti-lost terminal module respectively; and a smart control device, provided with a health parameter control module, an anti-lost control module, a display screen and a communication interface which is electrically connected to the health parameter control module and the anti-lost control module respectively. Each wearable device is connected to the smart control device in wireless manner by means of the communication interfaces, the health parameter test module is adapted for transmitting physical health parameter information to the smart control device by means of the communication interface of the wearable device, the health parameter control module is adapted for performing computational processing, storing and sending image information that reflects the health conditions of the body, and the display screen is adapted for displaying the image information; the anti-lost terminal module is provided with a location and time feedback module, and the anti-lost control module is provided with a parameter setting module and a location and time receiving module, the parameter setting module is adapted for setting a safety distance between the wearable devices, and the location and time feedback module is adapted for returning location and time information of the wearable devices to the location and time receiving module when a distance between the wearable devices is beyond the safety distance, and the display screen is adapting for displaying the location and time information.

Further, the anti-lost terminal module further comprises an out-of-range warning receiving module, a warning broadcast module, the anti-lost control module further comprises an out-of-range warning sending module, the out-of-range warning sending module is adapted for sending an out-of-range warning to the out-of-range warning receiving module when the distance between the wearable devices is beyond the safety distance, and warning broadcast module is adapted for broadcasting the out-of-range warning.

The health parameter test module comprises a blood pressure test module, an oxygen saturation test module, a pulse rate test module, a pedometer test module, a sweat analysis test module, an electroencephalogram test module, a body temperature test module and a blood sugar test module.

The wireless manner comprises Bluetooth connection, infrared connection, WIFI connection or Zigbee connection.

Advantageous effects of the present invention include: first, since the health parameter test module of each wearable device is wirelessly connected to the smart control device having the health parameter control module, and the physical health parameter information is translated into image information that reflects the health conditions of the body and finally is shown to the user by means of the display screen, thus the smart wearable device system of the present invention can test health parameters such as blood pressure, oxygen saturation, blood sugar, pulse rate, electroencephalogram, body temperature and sweat, and return the information to the user by image; second, since at least two wearable devices are configured, and a safety distance (e.g. 10 meters) between the wearable devices is set, if the distance therebetween is beyond the safety distance, the location and time information of the wearable devices will be returned to the location and time receiving module and displayed by the display screen, thus when multiple persons wear this device outdoors, it's timely to know the user's location and time who is out of the safety distance (namely the lost person), thereby the lost person will be found in time. Thus the present invention can prevent persons getting lost.

### BRIEF DESCRIPTION OF THE DRAWINGS

FIG. 1 is a schematic view of the system of the invention.

The labels includes:

1 wearable device, 11 heath parameter test module, 111 blood pressure test module, 112 oxygen saturation test module, 113 pulse rate test module, 114 pedometer test module, 115 sweat analysis test module, 116 electroencephalogram test module, 117 body temperature test module, 118 blood sugar test module, 12 anti-lost terminal module, 121 location and time feedback module, 122 out-of-range warning receiving module, 123 warning broadcast module, 13 communication interface, 2 smart control device, 21 health parameter control module, 22 anti-lost control module, 221 parameter setting module, 222 location and time receiving module, 223 out-of-range warning sending module, display screen 23, communication interface 24.

### DETAILED DESCRIPTION OF ILLUSTRATED EMBODIMENTS

For further understanding the features, technical solutions, purposes and functions of the present invention, and explaining the advantages and spirits of the invention, detailed descriptions for embodiments follow.

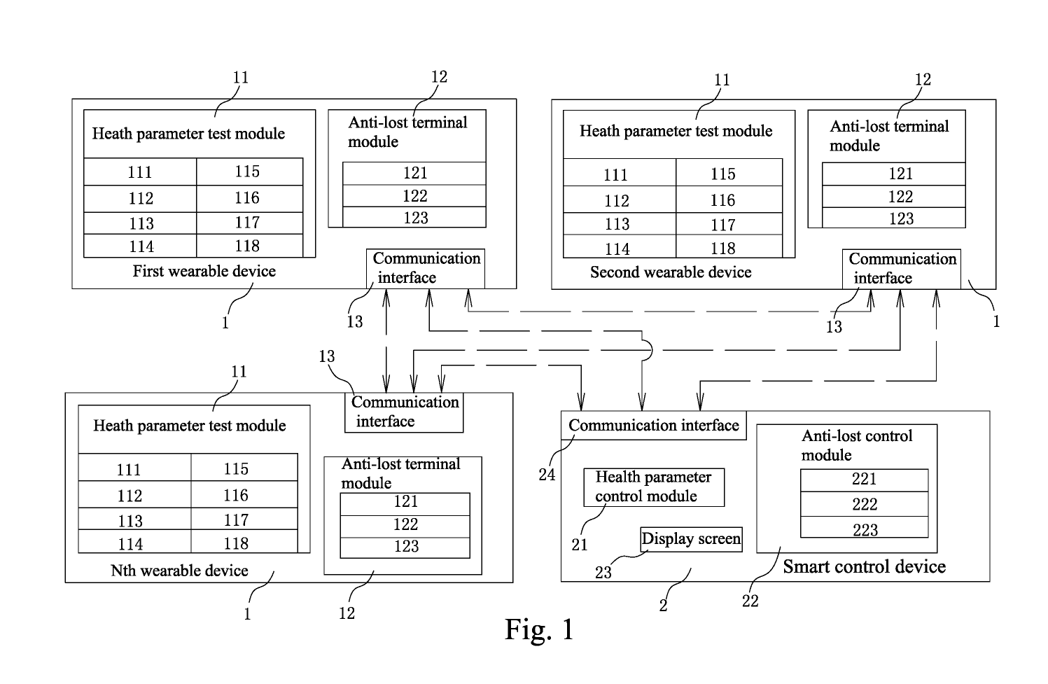

Referring to FIG. 1, the smart wearable device system includes at least two wearable devices 1 and a smart control device 2, as shown the wearable devices 1 are illustrated as a first wearable device 1, a second wearable device 1 and an Nth wearable device 1, and the smart control device 2 can be smart phone, personal PC, tablet PC, PDA or other mobile smart devices, preferably it is a smart phone. The wearable device 1 includes a health parameter test module 11, an anti-lost terminal module 12 and a communication interface 13 which is electrically connected to the health parameter test module 11 and the anti-lost terminal module 12 respectively, and the smart device 1 includes a health parameter control module 21, an anti-lost control module 22, a display screen 23 and a communication interface 24 which is electrically connected to the health parameter control module 21 and the anti-lost control module 22 respectively. Each wearable device 1 is connected to the smart control device 2 in wireless manner by means of the communication interfaces, and every two wearable devices 1 are connected to each other in wireless manner. After the health parameter test module 11 transmits physical health parameter information to the smart control device 2 by means of the communication interface, the health parameter control module 21 will receive the physical health parameter, analyze data, set parameters and send out instruction, store and send image information that reflects the health conditions of the body, and the information will be displayed by the display screen 23. The anti-lost terminal module 12 includes a location and time feedback module 121, and the anti-lost control module 22 includes a parameter setting module 221 and a location and time receiving module 222. Specifically, the parameter setting module 221 can set a safety distance (such as 10 meters) between the wearable devices 1, when the distance between the wearable devices 1 is beyond the safety distance, the location and time feedback module 121 returns the location and time information of the wearable devices 1 to the location and time receiving module 222, and then the information will be displayed by the display screen (23). Further, the wireless manner includes Bluetooth connection, infrared connection, WIFI connection or Zigbee connection.

As shown in FIG. 1, the anti-lost terminal module 12 further includes an out-of-range warning receiving module 122, a warning broadcast module 123, the anti-lost control module 22 further includes an out-of-range warning sending module 223. When the distance between the wearable devices 1 is beyond the safety distance, the out-of-range warning sending module 223 sends an out-of-range warning to the out-of-range warning receiving module 122, and then the out-of-range warning will be broadcast by the warning broadcast module 123, so that the user wearing the device will be warned to stay within the safety distance thereby preventing him from missing. Preferably the warning broadcast module 123 can be a speaker or a buzzer,

As shown in FIG. 1, the health parameter test module 11 includes a blood pressure test module 111, an oxygen saturation test module 112, a pulse rate test module 113, a pedometer test module 114, a sweat analysis test module 115, an electroencephalogram test module 116, a body temperature test module 117 and a blood sugar test module 118. Specifically, the pedometer test module 114 is used for testing running step and distance of the user, and the blood sugar test module 118 can perform the noninvasive testing by means of saliva or body fluid.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

What is claimed is:

1. A smart wearable device system, comprising:
at least two wearable devices (1), provided with a health parameter test module (11), an anti-lost terminal module terminal module (12) and a communication interface (13) which is electrically connected to the health parameter test module (11) and the anti-lost terminal module (12) respectively; and
a smart control device (2), provided with a health parameter control module (21), an anti-lost control module (22), a display screen (23) and a communication interface (24) which is electrically connected to the health parameter control module (21) and the anti-lost control module (22) respectively;
wherein each wearable device (1) is connected to the smart control device (2) in wireless manner by means of the communication interfaces, the health parameter test module (11) is adapted for transmitting physical health parameter information to the smart control device (2) by means of the communication interface (13) of the wearable device (1), the health parameter control module (21) is adapted for performing computational processing, storing and sending image information that reflects the health conditions of the body, and the display screen (23) is adapted for displaying the image information; the anti-lost terminal module (12) is provided with a location and time feedback module (121), and the anti-lost control module (22) is provided with a parameter setting module (221) and a location and time receiving module (222), the parameter setting module (221) is adapted for setting a safety distance between the wearable devices (1), and the location and time feedback module (121) is adapted for returning location and time information of the wearable devices (1) to the location and time receiving module (222) when a distance between the wearable devices (1) is beyond the safety distance, and the display screen (23) is adapting for displaying the location and time information.

2. The smart wearable device system according to claim 1, wherein the anti-lost terminal module (12) further comprises an out-of-range warning receiving module (122), a warning broadcast module (123), the anti-lost control module (22) further comprises an out-of-range warning sending module (223), the out-of-range warning sending module (223) is adapted for sending an out-of-range warning to the out-of-range warning receiving module (122) when the distance between the wearable devices (1) is beyond the safety distance, and warning broadcast module (123) is adapted for broadcasting the out-of-range warning.

3. The smart wearable device system according to claim 1, wherein the health parameter test module (11) comprises a blood pressure test module (111), an oxygen saturation test module (112), a pulse rate test module (113), a pedometer test module (114), a sweat analysis test module (115), an electroencephalogram test module (116), a body temperature test module (117) and a blood sugar test module (118).

4. The smart wearable device system according to claim 1, wherein the wireless manner comprises Bluetooth connection, infrared connection, WIFI connection or Zigbee connection.
